(19) United States
(12) Patent Application Publication
(10) Pub. No.: US 2008/0051677 A1
(43) Pub. Date: Feb. 28, 2008

(54) Method and apparatus for osteochondral autograft transplantation

(75) Inventor: Jeetendra Bharadwaj, Memphis, TN (US)

Correspondence Address: HAYNES AND BOONE, LLP, 901 Main Street, Suite 3100, Dallas, TX, 75202, US

(73) Assignee: Warsaw Orthopedic, Inc., Warsaw, IN (US)

(21) Appl. No.: 11/508,349

(22) Filed: Aug. 23, 2006

(51) Int. Cl. A61B 5/107; A61F 2/28

(52) U.S. Cl. 600/587; 623/14.12; 623/908

(57) ABSTRACT

An osteochondral transplantation procedure according to which a harvested graft is implanted in an area of a patient's body and is tracked relative to the area during the implantation. Images are displayed that correspond to the tracking.

### BACKGROUND

This invention relates to an improved osteochondral autograft transplantation procedure and apparatus, and more particularly, to such a procedure and apparatus in which a graft is prepared for a recipient socket.

The human knee consists of three bones—a femur, a tibia, and a patella—that are held in place by various ligaments. The corresponding condyles of the femur and the tibia form a hinge joint, and the patella protects the joint. Portions of the condyles, as well as the underside of the patella, are covered with an articular cartilage, which allow the femur and the tibia to smoothly glide against each other without causing damage.

The articular cartilage often tears, usually due to traumatic injury (often seen in athletes) and degenerative processes (seen in older patients). This tearing does not heal well due to the lack of nerves, blood vessels and lymphatic systems; and the resultant knee pain, swelling, and limited motion of the bone(s) must be addressed.

Damaged adult cartilages have historically been treated by a variety of surgical interventions including lavage, arthroscopic debridement, and repair stimulation, all of which provide less than optimum results.

Another known treatment involves removal and replacement of the damaged cartilage with a prosthetic device. However, prostheses have largely been unsuccessful since they are deficient in the elastic, and therefore in the shock-absorbing properties characteristic of the cartilage. Moreover, prostheses have not proven able to withstand the forces inherent to routine knee joint function.

In an attempt to overcome the problems associated with the above techniques, osteochondral autograft transplantation, also known as “mosaicplasty” has been used to repair articular cartilages. This procedure involves removing injured tissue from the damaged area and drilling one or more sockets in the underlying bone. A graft, or plug, consisting of healthy cartilage overlying bone, is obtained from another area of the patient, typically from a lower weight-bearing region of the joint under repair, or from a donor patient, and is implanted in each socket. It is extremely important that each plug fit in its socket in a precise manner and an embodiment of the present invention involves a technique for advancing the art in this respect.

### BRIEF DESCRIPTION OF THE DRAWING

FIG. 1 is an elevational view of a human knee with certain parts removed in the interest of clarity.

FIG. 2 is a diagrammatic view of an image guidance system according to an embodiment of the invention.

### DETAILED DESCRIPTION

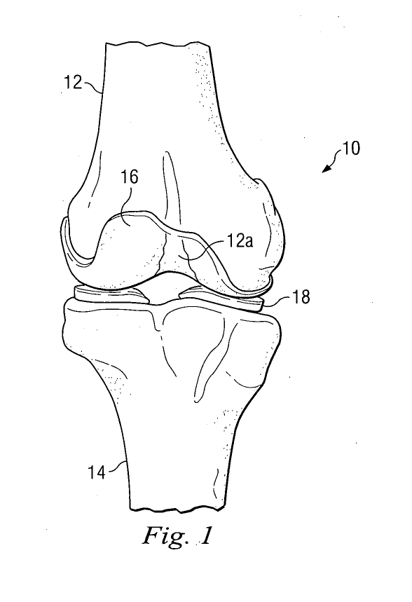

Referring to FIG. 1 of the drawing, the reference numeral 10 refers, in general, to a knee area of a human including a femur 12 and a tibia 14 whose respective condyles are in close proximity. A cartilage 16 extends over a portion of the condyle of the femur 12, and a meniscus 18 extends between the cartilage and the tibia 14. The patella, as well as the tendons, ligaments, and quadriceps that also form part of the knee, are not shown in the interest of clarity.

It will be assumed that a portion of the cartilage 16 extending over the condyle of the femur 12 has been damaged and resected by the surgeon, or has worn away, leaving a damaged area, or defect 12a. Referring to FIG. 1 of the drawing, the reference numeral 10 refers, in general, to a knee area of a human including a femur 12 and a tibia 14 whose respective condyles are in close proximity. A cartilage 16 extends over a portion of the condyle of the femur 12, and a meniscus 18 extends between the cartilage and the tibia 14. The patella, as well as the tendons, ligaments, and quadriceps that also form part of the knee, are not shown in the interest of clarity.

It will be assumed that a portion of the cartilage 16 extending over the condyle of the femur 12 has been damaged and resected by the surgeon, or has worn away, leaving a damaged area, or defect 12a. It will be also assumed that the surgeon has surgically removed areas of the bone below the damaged cartilage at the defect 12a so as to form a socket that is suited to receive a plug, or graft. The latter procedure can involve drilling a hole in the underlying bone to a predetermined depth that extends perpendicular to the surface of the femur 12 and examples of the specifics of this technique are disclosed in U.S. patent application No. (attorney's docket No. 31132.551) and U.S. Patent Application No. (attorney's docket No. 31132.555, and U.S. Patent application No. (attorney's docket No. 31132.556).

One or more grafts are harvested from another area of the patient/recipient, such as an undamaged non-load bearing area of the femur or tibia, or from a corresponding area of a donor, in accordance with known techniques. The graft is sized so as to be implantable in the socket at the defect 12a.

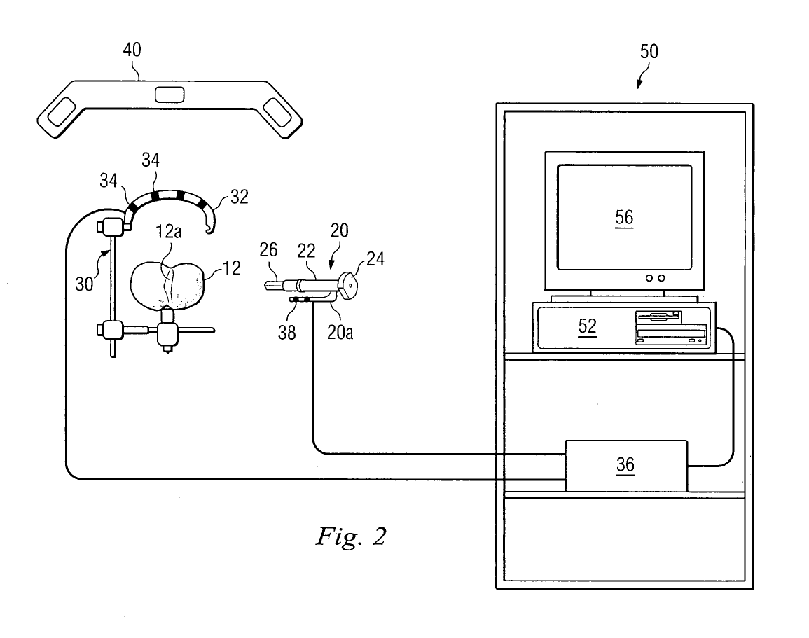

Referring to FIG. 2, the femur 12 is depicted in a substantially horizontal position, it being understood that it will be supported by a structure (not shown) such as a table, or the like. A tool 20 is provided that includes cylindrical body member 22 having a handle 24 disposed at one end, and a hollow tubular member 26 extending from the other end. A relatively sharp cutting edge is formed at the distal end of the member 26 for cutting the graft, in the form of a cylindrical plug, after which the cut graft is forced into the interior of the member 26. It will be assumed that the tool 20 also includes a mechanism for releasing the graft from the member 26. Examples of tools that can be used are disclosed in U.S. patent application Ser. No. 10/792,780, filed on Mar. 5, 2004 (now U.S. publication no. 2004/0176771, published Sep. 9, 2004); U.S. patent application Ser. No. 10/785,388, filed on Feb. 23, 2004 (now U.S. application publication no. 2004/0193154, published Sep. 30, 2004); U.S. patent application Ser. No. 10/984,497, filed Nov. 9, 2004; (now U.S. application publication no. 2005/0101962, published May 12, 2005); U.S. patent application Ser. No. 10/815,778, filed Apr. 2, 2004 (now U.S. application publication no. 2005/0222687, published Oct. 6, 2005); U.S. patent application Ser. No. 08/885,752, filed Jun. 30, 1997 (now U.S. Pat. No. 5,919,196 granted Jul. 6, 1999); U.S. patent application Ser. No. 08/797,973, filed Feb. 12, 1997 (now U.S. Pat. No. 5,921,987 granted Jul. 13, 1999); U.S. patent application Ser. No. 08/908,685, filed Aug. 7, 1997 (now U.S. Pat. No. 5,964,805, granted Oct. 12, 1999); U.S. patent application Ser. No. 08/774,799 filed Dec. 30, 1996 (now U.S. Pat. No. 6,007,496); U.S. patent application Ser. No. 09/187,283, filed on Nov. 5, 1998 (now U.S. Pat. No. 6,110,209, granted Aug. 29, 2000); U.S. patent application Ser. No. 09/425,337, filed Oct. 22, 1999 (now U.S. Pat. No. 6,306,142, granted Oct. 23, 2001); U.S. patent application Ser. No. 09/559,532, filed Apr. 28, 2000 (now U.S. Pat. No. 6,375,658, granted Apr. 23, 2002); U.S. patent application Ser. No. 09/118,680, filed Jul. 17, 1998 (now U.S. Pat. No. 6,395,011, granted May 28, 2002); U.S. patent application Ser. No. 09/624,689, filed Jul. 24, 2000 (now U.S. Pat. No. 6,440,141, granted Aug. 27, 2002); U.S. patent application Ser. No. 09/571,363, filed May 15, 2000 (now U.S. Pat. No. 6,488,033, granted Dec. 3, 2002); U.S. patent application Ser. No. 09/243,880, filed Feb. 3, 1999 (now U.S. Pat. No. 6,592,588, granted Jul. 15, 2003); U.S. patent application Ser. No. 10/004,388, filed Oct. 23, 2001 (now U.S. Pat. No. 6,767,354, granted Jul. 27, 2004); U.S. patent application Ser. No. 10/084,490, filed Feb. 28, 2002 (now U.S. Pat. No. 6,852,114, granted Feb. 8, 2005); U.S. patent application Ser. No. 10/665,152, filed on Sep. 22, 2003 (now U.S. publication no. 2004/0059425, published Mar. 25, 2004); U.S. patent application Ser. No. 10/638,489, filed on Aug. 12, 2003 (now U.S. publication no. 2004/0034437, published Feb. 19, 2004); U.S. patent application Ser. No. 10/443,893, filed on May 23, 2003 (now U.S. publication no. 2004/0039400, published Feb. 26, 2004); U.S. patent application Ser. No. 10/947,217, filed on Sep. 23, 2004 (now U.S. publication no. 2006/0060209, published Mar. 23, 2006). The disclosures of each of these patents are incorporated herein by reference.

Inasmuch as the surface of the defect 12a is curved, and the socket extends perpendicularly to the surface, it is a challenge to insure that the graft is inserted in the socket at the defect 12a precisely perpendicular to the surface and precisely aligned with the socket. To this end an image guiding system is provided and is shown, in general, by the reference numeral 28 in FIG. 2.

The image guiding system 28 includes a conventional clamping device such as a Mayfield clamp assembly 30, which is clamped around the leg. A reference frame 32 is mounted on the assembly 30 in a spatial relation to the femur 12, and supports a series of tracking devices, or emitters, 34, preferably in the form of light emitting diodes (“LEDs”).

It is understood that, prior to the procedure, an image data set, usually generated by a CAT scanner, or the like, which image has reference points that have a fixed spatial relation to the femur 12. The image data is stored in a digitizer control unit 36 that will be described later, and the emitters 34 generate signals representing the position of the various reference points.

An arm 20a is mounted on the tool 20 and a series of tracking devices, or emitters 38, also preferably in the form of LEDs, are provided on the arm for providing a positive emission.

A position sensing unit, in the form of a sensor array 40, is supported relative to the assembly 30 and the femur 12 in any conventional manner. When the tool 20 is brought in proximity to the femur 12 during the procedure, the array 40 functions to track the position of the emitters 34 and 38 so that it can identify, during the procedure, the relative position of each of the above reference points and the tool.

A workstation 50 is provided that includes the unit 36 along with a processor 52 such as a PC, a CPU, a server, or the like. The unit 36 is connected to the emitters 34 and 38, and to the processor 52 for modifying the above stored image data set according to the identified relative position of each of the reference points during the procedure, as identified by the sensor array 40. The processor 52 is connected to a monitor 56 that generates a displaced image data set representing the position of the femur 12 and the tool 20 in three dimensions.

Since the image guiding system 28 is well known in the art it will not be described in any further detail. As examples of the latter art, reference is made to the following U.S. patents: U.S. re-issue patent application Ser. No. 10/423,332 filed Apr. 24, 2003 (now U.S. patent no. RE39,133 granted Jun. 13, 2006); U.S. patent application Ser. No. 10/289,869 filed Nov. 7, 2002 (now U.S. Pat. No. 7,007,699 granted Mar. 7, 2006); U.S. patent application Ser. No. 10/198,324 filed Jul. 18, 2002 (now U.S. Pat. No. 6,978,166 granted Dec. 20, 2005); U.S. patent application Ser. No. 10/177,739 filed Jun. 21, 2002 (now U.S. Pat. No. 6,920,347 granted Jul. 19, 2005); U.S. patent application Ser. No. 10/223,847 filed Aug. 19, 2002 (now U.S. Pat. No. 6,892,090 granted May 10, 2005); U.S. patent application Ser. No. 09/992,546 filed Nov. 6, 2001 (now U.S. Pat. No. 6,796,988 granted Sep. 28, 2004); U.S. patent application Ser. No. 09/461,241 filed Dec. 16, 1999 (now U.S. Pat. No. 6,754,374 granted Jun. 22, 2004; U.S. patent application Ser. No. 09/795,126 filed Mar. 1, 2001 (now U.S. Pat. No. 6,725,080 granted Apr. 20, 2004); U.S. patent application Ser. No. 10/047,927 filed Jan. 14, 2002 (now U.S. Pat. No. 6,669,635 granted Dec. 30, 2003); U.S. patent application Ser. No. 09/873,604 filed Jun. 4, 2001 (now U.S. Pat. No. 6,636,757 granted Oct. 21, 2003); U.S. patent application Ser. No. 09/299,567 filed Apr. 27, 1999 (now U.S. Pat. No. 6,553,152 granted Apr. 22, 2003); U.S. patent application Ser. No. 09/464,180 filed Dec. 16, 1999 (now U.S. Pat. No. 6,540,668 granted Apr. 1, 2003); U.S. patent application Ser. No. 09/545,092 filed Apr. 7, 2000 (now U.S. Pat. No. 6,535,756 granted Mar. 18, 2003); U.S. patent application Ser. No. 09/557,004 filed Apr. 20, 2000 (now U.S. Pat. No. 6,491,699 granted Dec. 10, 2002); U.S. patent application Ser. No. 09/105,067 filed Jun. 26, 1998 (now U.S. Pat. No. 6,490,467 granted Dec. 3, 2002); U.S. patent application Ser. No. 09/428,722 filed Oct. 28, 1999 (now U.S. Pat. No. 6,474,341 granted Nov. 5, 2002); U.S. patent application Ser. No. 09/274,972 filed Mar. 23, 1999 (now U.S. Pat. No. 6,470,207 granted Oct. 22, 2002); U.S. patent application Ser. No. 09/598,975 filed Jun. 21, 2000 (now U.S. Pat. No. 6,434,507 granted Aug. 13, 2002); U.S. patent application Ser. No. 09/398,313 filed Sep. 20, 1999 (now U.S. Pat. No. 6,434,415 granted Aug. 13, 2002); U.S. patent application Ser. No. 09/803,977 filed Mar. 13, 2001 (now U.S. Pat. No. 6,402,762 granted Jun. 11, 2002); U.S. patent application Ser. No. 09/429,569 filed Oct. 28, 1999 (now U.S. Pat. No. 6,381,485 granted Apr. 30, 2002); U.S. patent application Ser. No. 09/428,720 filed Oct. 28, 1999 (now U.S. Pat. No. 6,379,302 granted Apr. 30, 2002); U.S. patent application Ser. No. 09/209,248 filed Dec. 10, 1998 (now U.S. Pat. No. 6,348,058 granted Feb. 19, 2002); U.S. patent application Ser. No. 09/555,885 filed Oct. 8, 1999 (now U.S. Pat. No. 6,340,363 granted Jan. 22, 2002); U.S. patent application Ser. No. 09/382,594 filed Aug. 25, 1999 (now U.S. Pat. No. 6,253,210 granted Jun. 26, 2001); U.S. patent application Ser. No. 08/809,404 filed Oct. 5, 1995 (now U.S. Pat. No. 6,236,875 granted May 22, 2001); U.S. patent application Ser. No. 09/429,568 filed Oct. 28, 1999 (now U.S. Pat. No. 6,235,038 granted May 22, 2001); U.S. patent application Ser. No. 09/148,498 filed Sep. 4, 1998 (now U.S. Pat. No. 6,226,548 granted May 1, 2001); U.S. patent application Ser. No. 09/296,251 filed Apr. 22, 1999 (now U.S. Pat. No. 6,190,395 granted Feb. 20, 2001); U.S. patent application Ser. No. 08/623,956 filed Mar. 29, 1996 (now U.S. Pat. No. 6,167,145 granted Dec. 26, 2000); U.S. patent application Ser. No. 09/106,109 filed Jun. 29, 1998 (now U.S. Pat. No. 6,118,845 granted Sep. 12, 2000); U.S. design patent application serial no. 29/070,111 filed Apr. 30, 1997 (now U.S. design patent no. D422,706 granted Apr. 11, 2000); U.S. design patent application serial no. 29/079,326 filed Nov. 3, 1997 (now U.S. design patent no D420,132 granted Feb. 1, 2000); U.S. patent application Ser. No. 08/971,126 filed Nov. 20, 1997 (now U.S. Pat. No. 6,021,343 granted Feb. 1, 2000); and U.S. design patent application serial no. 50,227 filed Feb. 12, 1996 (now U.S. design patent no. D387,427 granted Dec. 9, 1997), U.S. patent application serial no. 909,097 filed Jul. 2, 1992 (now U.S. Pat. No. 5,383,454 granted Jan. 24, 1995); PCT Application No. PCT/US94/04530 (Publication No. WO 94/24933); PCT Application No. PCT/US95/12894 (Publication No. WO 96/11624), to Bucholz et al.; U.S. patent application serial no. 543,516 filed Oct. 16, 1995 (now U.S. Pat. No. 5,851,183 granted Dec. 22, 1998); U.S. patent application serial no. 524,981 filed Sep. 7, 1995 (now U.S. Pat. No. 5,871,445 granted Feb. 16, 1999); U.S. patent application serial no. 477,561 filed Jun. 7, 1995 (now U.S. Pat. No. 5,891,034 granted Apr. 6, 1999); U.S. patent application Ser. No. 09/848,267 filed May 4, 2001 (now U.S. Pat. No. 6,708,184 granted Mar. 16, 2004). The disclosures of each patent are incorporated herein by reference.

To initiate the harvesting procedure, the tool 20 is brought to an area of the femur close to, but spaced from, the defect 12a. The above-mentioned cutting edge of the member 26 is positioned over the latter area and then advanced further towards the femur 12 until the cutting edge slices through the layer of cartilage. The manual force is continued and could be increased as necessary so that the cutting edge also cuts through the condyle below the cartilage until the desired depth of cut is attained. During this time, the severed graft, including the cartilage and condyle next to the cartilage, enter the hollow distal end portion of the member 26 and are retained in the latter member. When the desired depth of cut is attained, the tool 20 is manipulated as necessary to completely sever the corresponding end of the condyle, thus forming a graft extending in the interior of the member 26.

The tool 20 is then moved to the immediate vicinity of the defect 12 and the harvested graft is then ejected, or otherwise removed, from the tool 20 and implanted in the above-mentioned socket in the defect 12a. To this end, the controller 36 responds to the signals from the emitters 34 and 38, and the sensor array 40, and modifies the above-mentioned stored image data set according to the identified relative position of each of the reference points during the procedure. The processor 52 may then generate an image data set representing the relative position of the femur 12 and the tool 20 during the procedure, which is displayed on the monitor 56, thus enabling the surgeon to determine the relative positions of the tool 20 and the femur 12 in real time and in three dimensions.

By viewing the monitor 56 during the procedure in real time, the surgeon can manipulate the tool 20 so that it extends perpendicularly to the surface of the femur when the graft is harvested, when the tool 20 is advanced to the socket, and when the graft is implanted into the socket, all in three dimensions. As a result, the contour of the cartilage of the graft will match the contour of the cartilage surrounding the defect 12a.

It should be emphasized that the harvesting of the graft, the forming of the recipient opening, and the implanting of the graft have all been described fairly generally above and are disclosed with more detail in U.S. application Ser. No. 11/340,024 filed on Jan. 26, 2006; U.S. application Ser. No. 11/338,926 filed on Jan. 25, 2006; U.S. application Ser. No. 11/339,194 filed on Jan. 25, 2006; U.S. application Ser. No. 11/317,985 filed Dec. 23, 2005; U.S. application Ser. No. 11/340,884 filed on Jan. 27, 2006; U.S. application Ser. No. 11/343,156 filed on Jan. 30, 2006; U.S. application Ser. No. 11/339,694 filed Jan. 25, 2006; and also in (attorney docket Nos. 31132.498, 31132.552, 1132.553, 31132.551, 1132.555, and 31132.556) the disclosures of each of which are incorporated herein by reference.

It is also understood that an overlay system, such as the one disclosed in the assignee's U.S. Patent application (attorneys' docket number 31132.556) can be used to assist in positioning the tool 20 relative to the femur 12 during the above procedure.

It is also understood that, during the above procedure, any of the meniscus 18 (FIG. 1) or related tendons, ligaments and quadriceps are removed or pushed aside as necessary to permit access to the above area to permit the harvesting of the graft and/or the cutting of the socket, and/or the implantation of the graft.

As discussed in general above, it is understood that, several days before surgery, the images of the patient's knee anatomy can be prepared. The images can be downloaded into the processor 52 and the system creates reference points in three-dimensions of the femur 12, including the defect 12a. The images can be rotated, enlarged, flipped, angled, or manipulated in a variety of manners. This allows the surgeon to accurately pre-plan the surgical procedure including determining the number and size of the graft(s) and corresponding sockets in the femur 12, and therefore the size of the tool 20.

### Variations

1. The tool 20 can be used to harvest the graft, retain the graft, and/or implant the graft in the socket in the defect, or separate tools can be provided for each function while the surgeon can monitor each function on the monitor 30.

2. The shape and dimensions of the tool 20 and therefore the graft can vary within the scope of the invention. For example, the tubular member 26, as well as its cutting edge 26a, and therefore the graft, can have a rectangular cross section such as disclosed in U.S. Application No. (Attorney's docket No. 31132.436), the disclosure of which is incorporated herein by reference.

3. Although only one socket in the defect 12a is described above, it is understood that a plurality of sockets can be formed in the defect 12a which are filled with a corresponding number of grafts.

4. The spatial references mentioned above, such as “upper”, “lower”, “under”, “over”, “between”, “outer”, “inner” “surrounding” and “horizontal” are for the purpose of illustration only and do not limit the specific orientation or location of the members described above.

Those skilled in the art will readily appreciate that many other variations and modifications of the embodiment described above can be made without materially departing from the novel teachings and advantages of this invention. Accordingly, all such variations and modifications are intended to be included within the scope of this invention as defined in the following claims. In the claims, means-plus-function clauses are intended to cover the structures described herein as performing the recited function and not only structural equivalents, but also equivalent structures.

What is claimed is:

1. An osteochondral transplantation procedure comprising:
harvesting a graft;
implanting the graft in an area of the patient's body;
tracking the position of the graft relative to the area during the step of implanting;
responding to the step of tracking and providing corresponding images of the graft and the area in three dimensions; and
displaying the images.

2. The procedure of claim 1 wherein the step of harvesting comprises cutting the graft from another area of the patient's body or from a donor patient's body.

3. The procedure of claim 2 wherein the step of harvesting is done with a tool, and further comprising the step of retaining the graft in the tool after the step of harvesting, and releasing the graft from the tool during the step of implanting.

4. The procedure of claim 2 wherein the graft is harvested from a first area of the femur of the patient's body and wherein the graft is implanted in a second area of the femur of the patient's body.

5. The procedure of claim 2 wherein the graft is harvested from a donor patient's body.

6. The procedure of claim 1 further comprising forming a socket in the patient's body for receiving the graft.

7. The procedure of claim 6 wherein the socket is formed in the femur of the patient's body.

8. The procedure of claim 1 further comprising storing an image data set that includes reference points that have a fixed spatial relation to the femur, emitting a first set of signals representing the position of the various reference points, and emitting a second set of signals representing the position of the tool.

9. The procedure of claim 8 further comprising sensing the first and second set of signals, processing the signals, and generating an image data set representing the relative position of the femur and the tool.

10. An osteochondral transplantation procedure comprising:
cutting a graft from an area of the patient's body;
tracking the position of the tool relative to the area during the step of cutting;
responding to the step of tracking and providing corresponding images of the graft and the area in three dimensions; and
displaying the images.

11. The procedure of claim 10 wherein the area is in the femur of the patient's body.

12. The procedure of claim 10 wherein the step of cutting is done with a tool, and further comprising the steps of retaining the graft in the tool after the step of cutting, and then implanting the graft in a socket in the patient's body.

13. The procedure of claim 10 further comprising storing an image data set that includes reference points that have a fixed spatial relation to the femur, emitting a first set of signals representing the position of the various reference points, and emitting a second set of signals representing the position of the tool.

14. The procedure of claim 13 further comprising sensing the first and second set of signals, processing the signals, and generating an image data set representing the relative position of the femur and the tool.

15. An osteochondral procedure for transplanting a graft in an area of a patient's body, the procedure comprising:
retaining the graft in a tool;
tracking the position of the tool relative to the area;
responding to the step of tracking and providing corresponding images of the graft and the area in three dimensions; and
displaying the images.

16. The procedure of claim 15 wherein the area is in the femur of the patient's body.

17. The procedure of claim 15 further comprising harvesting the graft from another area of the patient's body or from a donor patient's body.

18. The procedure of claim 17 wherein the step of harvesting comprises cutting the graft from the other area with the tool.

19. The procedure of claim 15 further comprising storing an image data set that includes reference points that have a fixed spatial relation to the femur, emitting a first set of signals representing the position of the various reference points, and emitting a second set of signals representing the position of the tool.

20. The procedure of claim 19 further comprising sensing the first and second set of signals, processing the signals, and generating an image data set representing the relative position of the femur and the tool.
